(19) United States
(12) Plant Patent Application Publication
(10) Pub. No.: US 2013/0055475 P1
(43) Pub. Date: Feb. 28, 2013

(54) Grapevine 'IFG Nine'

(50) ;

(71) Applicant: David Cain, Bakersfield, CA (US)

(72) Inventor: David Cain, Bakersfield, CA (US)

(73) Assignee: International Fruit Genetics, LLC, Bakersfield, CA (US)

(21) Appl. No.: 13/199,228

(22) Filed: Aug. 24, 2011

(51) Int. Cl. A01H 5/00

(52) U.S. Cl. PLT/205

(57) ABSTRACT

This invention is a new and distinct grapevine variety named ‘IFG Nine’ which is characterized by producing naturally large light red, firm, narrow ellipsoid to somewhat cylindrical seedless grapes borne on large clusters. The fruit ripen and are harvestable in early to mid-September.

### LATIN NAME OF THE GENUS AND SPECIES CLAIMED

Vitis vinifera

### VARIETY DENOMINATION

‘IFG Nine’

### BACKGROUND OF THE INVENTION

The new and distinct grapevine described and claimed herein originated from a hand pollinated cross of the Redglobe variety (U.S. Plant Pat. No. 4,787 expired) and the Princess variety (non-patented) hybridized in May 2001. The seeds were subsequently germinated and the resulting plants were planted in the field in April 2002. The present variety of grapevine was selected as a single plant in August 2003 and was first asexually propagated by hardwood cuttings in December 2003 near Delano, Kern County, Calif. The resulting propagules were planted during April 2004 near Delano, Kern County, Calif. and were found to reproduce true-to-type through at least three generations of asexual reproduction.

### BRIEF SUMMARY OF THE INVENTION

The new grapevine ‘IFG Nine’ is characterized by producing naturally large, narrow ellipsoid to somewhat cylindrical, red seedless berries which ripen with or slightly before the Crimson variety. Berries have crisp texture, excellent eating quality and are able to develop bright red skin color more readily than the Crimson variety.

To the inventor's knowledge, the known varieties to which the new grapevine variety is most similar are the Crimson Variety (unpatented) and the Sheegene-1 (U.S. Plant Pat. No. 18,937). The ‘IFG Nine’ can be distinguished from the Crimson variety by having much larger natural berry size. The ‘IFG Nine’ does not require the use of gibberellic acid to attain acceptable commercial berry size while the Crimson variety requires gibberellic acid applications and/or girdling to attain acceptable size. ‘IFG Nine’ also colors more easily than the Crimson variety and does not require the application of ethrel or other color enhancing chemicals to attain commercially acceptable red color. The ‘IFG Nine’ can be distinguished from the Sheegene-1 variety by having a slightly more narrow ellipsoid to cylindrical shaped berry as opposed to the more ovate to obtuse ovate berry shape of Sheegene-1. The ‘IFG Nine’ has a larger and less compact cluster than the Sheegene-1. The ‘IFG Nine’ cluster weight is about 1592 grams and has about 255 berries per bunch as opposed to 719 grams with 155 berries per bunch for the Sheegene-l. The berry weight of ‘IFG Nine’ is somewhat smaller than Sheegene-1 being 6.9 to 8.4 grams per berry as opposed to about 11.4 grams for Sheegene-1.

‘IFG nine’ can be distinguished from its parent, the Redglobe variety by producing seedless rather than seeded berries having a smaller more narrow ellipsoid to cylindrical shape as opposed to the round shape of Redglobe. ‘IFG Nine’ can be distinguished from its parent the Princess variety by having red rather than white skin coloration and by its later ripening period. ‘IFG Nine’ can further be distinguished based on the characteristics described below.

### BRIEF DESCRIPTION OF THE FIGURE

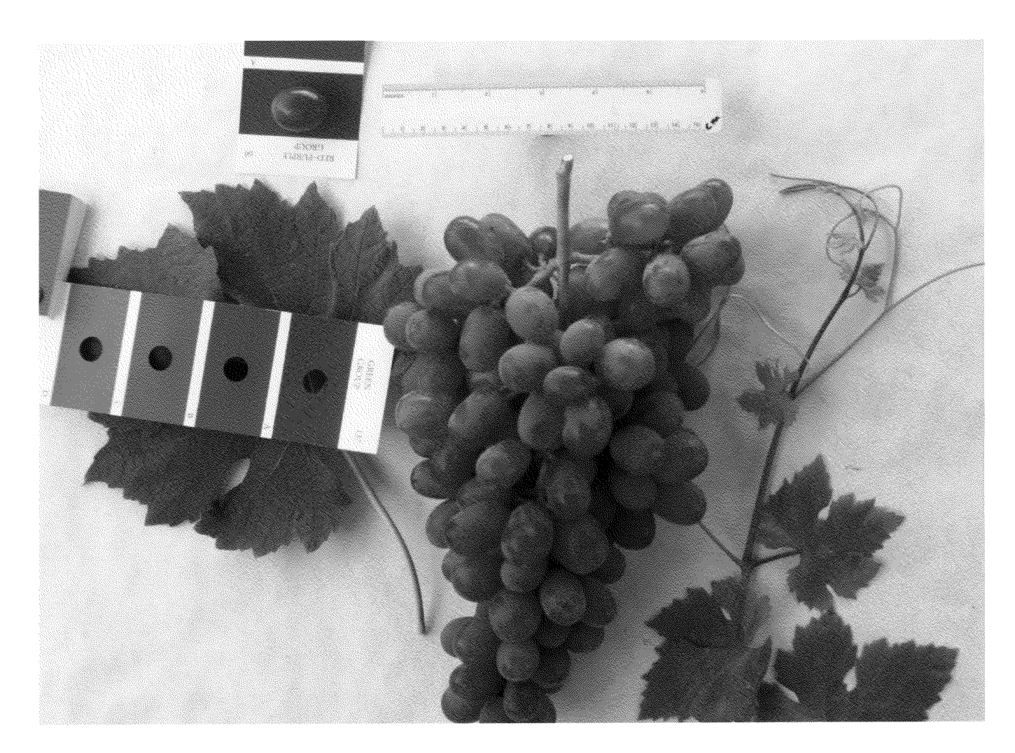

The accompanying photographic illustration in FIG. 1 illustrates in full color ‘IFG Nine’. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (Vitis spp.) (1983) which was developed in collaboration with the Office International de la Vigne et du Vin (OIV) and the International Union for the Protection of New Varieties of Plants (UPOV).

The descriptive matter which follows pertains to ‘IFG Nine’ plants grown in the vicinity of Delano, Kern County, Calif. during 2009, and 2010 and is believed to apply to plants of the variety grown under similar conditions of soil and climate elsewhere:
Vine:
   General.—Size — Large Vigor — Vigorous Density of foliage — Dense Productivity — Productive Root stock — Own root Training method — Typically spur pruned leaving 2 bud spurs.
   Trunk.—Trunk diameter of 4-year-old vines at 30 cm above the soil line — 5.5 cm Shape — Medium Straps — Split Surface texture — Medium Inner bark color — Can be any of the following colors; Greyed-orange; 177A and 177B.
Shoots:
   Young shoot.—Form of tip — Wide open Distribution of anthocyanin coloration of tip — Piping (striped) Intensity of anthocyanin coloration of tip — Weak Density of prostrate hairs of tip — Medium Density of erect hairs of tip — Absent Color — 144A.
   Woody shoot (Mature canes).—Shape — Stocky Internode length — Medium; About 12.4 cm Width at node — About 13 mm Cross section — Circular Surface — Striate Main color — Can be any of the following colors; Greyed Orange; 165B and C, and 172C Density of erect hairs of nodes — None or very sparse Density of erect hairs on internodes — None or very sparse Growth of axillary shoots — Medium; Approximately 10.6 cm.
   Flowering shoot.—Vigor during flowering — Strong Attitude during flowering on shoots not tied — Semi-erect Color — dorsal side of internodes — Green with Red stripes Color — ventral side of internodes — Green with Red stripes Color — dorsal side of nodes — Green with Red stripes Color — ventral side of nodes — Green with Red stripes Density of prostrate hairs of nodes — None Density of erect hairs of nodes — Very sparse Density of prostrate hairs on internode — Sparse Density of erect hairs on internode — None Anthocyanin coloration of buds — Absent.
   Tendrils.—Distribution on the shoot (at full flowering) — Discontinuous Length of Tendril — Medium-Long; about 22cm Thickness — Thick Color — Can be any of the following colors; Yellow-green; 144A, and 145A Form — Trifurcated-Quad furcated Number of consecutive tendrils — up to 2.
Leaves:
   Young leaves.—Color of upper surface of first four distal unfolded leaves — Copper yellow Average intensity of anthocyanin coloration of six distal leaves prior to flowering — Absent or very weak Density of prostrate hairs between veins (lower surface) — Absent Density of prostrate hairs on veins (lower surface) — Sparse Density of erect hairs between veins (lower surface) — Absent Density of erect hairs on veins (lower surface) — Very sparse.
   Mature leaves.—Average length — About 11.6 cm Average width — About 14.9 cm Mature leaf size — Medium Shape of blade — Pentagonal Number of lobes — 5 Anthocyanin coloration of main veins on upper side of blade — Absent Mature leaf profile — V-shaped Blistering surface of blade upper surface — Weak Leaf blade tip — In the plane of the leaf Undulation of margin — Pronounced Thickness — Thick-Medium Undulation of blade between main and lateral veins — Absent. Shape of teeth — Mixture of both sides straight and both sides convex Length of teeth — Short to Medium Ratio length/width of teeth — Small Shape of upper lateral sinuses — Lobes slightly overlapping Depth of upper lateral sinuses — Medium General shape petiole sinus — Lobes slightly overlapping Shape of base of upper leaf sinuses — V-shaped Tooth at petiole sinus — Present on about 50% of first emerging leaves Density of prostrate hairs between veins on lower surface of blade — Sparse Density if erect hairs between veins on lower surface of blade — Sparse Density of prostrate hairs on main veins on lower surface of blade — None or very sparse Density of erect hairs on main veins on lower surface of blade — Sparse Density of prostrate hairs on main veins on upper surface of blade — Sparse Density of erect hairs on main veins on upper surface of blade — None or very sparse Autumn coloration of leaves — Leaves can be a single color or combination of colors, in a mottled pattern or on the edges of the leaves; Greyed-yellow; 162 A and B.
   Upper surface.—Color — Can be any of the following colors; Green; 137A, and B, and C Anthocyanin coloration of main veins — Absent Surface appearance — Semi-glossy Blistering surface of blade — Weak.
   Lower surface.—Color — Can be any of the colors; Green; 144A, and 146B Anthocyanin coloration of main veins (lower surface) — Absent Glossiness — Weak-Medium Surface texture — Smooth Surface appearance — Semi-glossy.
   Petiole.—Length — About 9.2 cm Length of petiole compared to middle vein — Equal Density of prostrate hairs on petiole — Sparse Density of erect hairs on petiole — None.
   Buds.—Bud fruitfulness — Basal: Mostly fruitful Position of first fruitful shoot on previous season cane — 1^{st} to 2^{nd} node Time of bud burst — Late, Mar. 17, 2010.
Flowers:
   General.—Flower sex — Hermaphrodite Length of first inflorescence — Long; About 19.2 cm long by 13.0 cm wide Position of first flowering and fruiting node — 3^{rd}- 4^{th} Number of inflorescence per flowering shoot — 1.1 to 2 Time of bloom — Late as compared with similar varieties in the growing area of Delano, Calif. Date of full bloom — May 18, 2010.
Fruit:
   General.—Ripening period — Late; Approximately Sep. 25, 2009 Use — Fresh market Keeping quality — Good Resistance to — Insects: Average typical of Vitis vinifera species. Diseases: Average typical of Vitis vinifera species Shipping quality — Good Refractometer test — Soluble solids: About 19.6 Brix Brix/acid — About 47.5 Titratable acidity — About 0.41 Juice pH — About 4.19.
   Cluster.—Mature cluster length (peduncle excluded) — About 29.1 cm Mature cluster width — About 18.3 cm Mature cluster weight — About 1592 g Bunch density — Medium Number of berries — About 255 Form — Conical.
   Peduncle.—Lignification of peduncle — Strong Length of peduncle — Approximately 5.3 cm.
   Berry.—Uniformity of size — Uniform Single berry weight — About 6.9 g natural; to about 8.4 g when treated with gibberellic acid Shape — narrow ellipsoid to somewhat cylindrical Seeds — Absent may have an occasional noticeable seed trace Cross section — Circular Berry dimensions — Longitudinal axis: About 27.4 mm. Horizontal axis: About 19.7 mm Berry firmness — Medium Particular flavor — Neutral Bloom (cuticular wax) — Strong Berry separation from pedicel — Difficult Skin color (without bloom) — Red-purple; 60A.
   Skin.—Thickness — Medium Texture — Medium Reticulation — Absent Tenacity — Tenacious to flesh.

What is claimed:

1. A new and distinct variety of grapevine as herein illustrated and described.
